(19) United States
(12) Patent Application Publication
(10) Pub. No.: US 2025/0269089 A1
(43) Pub. Date: Aug. 28, 2025

(54) SELF-HEALING INJECTABLE SUPER-LUBRICATING HYDROGELS AND BIOMEDICAL APPLICATIONS THEREOF

(71) Applicant: Oligo Médic inc., Laval (CA)

(72) Inventors: Vahid ADIBNIA, Laval (CA); Abdellatif CHENITE, Kirkland (CA); Elias ASSAAD, Montréal (CA); Sam Alexandre SELMANI, Montréal (CA)

(21) Appl. No.: 18/858,026

(22) PCT Filed: Apr. 20, 2023
(86) PCT No.: PCT/CA2023/050532

§ 371 Date: Oct. 18, 2024

Related U.S. Application Data
(60) Provisional application No. 63/363,326, filed on Apr. 21, 2022.

(51) Int. Cl. A61L 27/52; A61K 9/06; A61K 31/167; A61K 38/06; A61K 47/36; A61L 27/20

(52) U.S. Cl. CPC A61L 27/52; A61K 9/06; A61K 31/167; A61K 38/063; A61K 47/36; A61L 27/20

(57) ABSTRACT

It is provided a lubricating hydrogel composition comprising an amine-bearing biopolymer reversibly cross-linked to an oxidized aldehyde-bearing polysaccharide, the hydrogel forming a protective hydration layer on negatively charged surfaces, providing the surface with lubrication properties similar to that in the healthy human joints and protect the surface against frictional wear.

### CROSS-REFERENCE TO RELATED APPLICATIONS

The present application claims benefit of U.S. Provisional Application No. 63/363,323 filed Apr. 21, 2022, the content of which is hereby incorporated by reference in their entirety.

### TECHNICAL FIELD

It is provided a lubricating hydrogel composition comprising an amine-bearing biopolymer reversibly cross-linked to an oxidized aldehyde-bearing polysaccharide.

### BACKGROUND

Biological systems require constant lubrication to maintain their function. Lubrication in human body occurs on the hair, skin, eyes, joints, gastrointestinal tract and reproductive tract. Lubrication in the human body is maintained through highly hydrophilic macromolecules, namely mucin, glycoproteins, phospholipids and hyaluronic acid, which work in synergy to provide the necessary lubrication properties on biological surfaces (Adibnia et al., 2020, Progress in Polymer Science, 110:101298). The primary role of these macromolecules in a lubricating media is to enhance the distribution and retention of water at the liquid-solid interface though formation of nanometric hydration layers (Adibnia et al., 2021, ACS Nano, 15:8953-8964; Adibnia et al., 2020, Journal of the American Chemical Society, 142:14843-14847). Improper lubrication, resulting from depletion of biological media from lubricating macromolecules, leads to severe health problems with short- and long-term consequences, such as osteoarthritis, dry eye, dry mouth (Cooper et al., 2018, Biomaterials, 181:210-226). Therefore, hydrated macromolecules that can be administered locally or systematically to lubricate biological surfaces are of significant importance.

Chitosan is a natural polysaccharide of D-glucosamine and N-acetyl-D-glucosamine units linked by (1-4) glycosidic bonds. It is derived by alkaline deacetylation of chitin, a component of the exoskeleton of crustaceans, the cuticles of insects and the cell walls of fungi (Pella et al., 2018, Carbohydrate Polymers, 196:233-245). Although chitosan itself is not soluble at neutral pH values, one of its derivatives, carboxylated chitosan, is a highly hydrophilic polymer that can be soluble in aqueous media in a wide range of physiologically relevant pH values (Wang et al., 2020, International Journal of Molecular Sciences, 21:487).

Glycogen, on the other hand, is another natural polysaccharide made of glucose monomers joined by (1-4) glycosidic linkages with branching, on average, every 13 monomers through (1-6) glycosidic bonds. The highly branched structure of this polysaccharide results in a globular macromolecule with a diameter of 30-100 nm (depending on the plant or animal source) (Nickels et al., 2016, Biomacromolecules, 17:735-743). A sweet corn-derived variation of glycogen has been extracted to yield a narrow size distribution about 70 nm diameter (U.S. Pat. No. 9,737,608). These NPs exhibit outstanding water retention, due to the abundance of hydroxyl groups forming hydrogen bonds with water in their tightly packed branched nanostructure. This excellent water retention property is the primary reason that glycogen nanoparticles (NPs) are known as the most efficient lubricating biomacromolecule in the literature.

Despite this outstanding lubrication properties, for some biomedical applications, such as lubrication of synovial joints, glycogen nanoparticles are too small to be retained in the biological media and their clearance time is expected to be short if administered directly to the media. For example, the synovial joints are estimated to have a pore size of 66-118 nm, which is sufficiently large for extrusion of the NPs (Sabaratnam et al., 2005, Journal of Physiology, 567:569-581). Therefore, to increase the residence time of these NPs in biological media and their therapeutic effect, it is beneficial to conjugate them on other hydrophilic polymers, potentially forming hydrogels that degrade slowly.

Hydrogels, cross-linked water-swollen networks of hydrophilic polymers, are attractive soft materials for biomedical applications, such as biolubrication, regenerative medicine and drug delivery, mainly because of their ability to mimic the microstructure and physicochemical properties of various biological media. Depending on the nature of physicochemical bonds in the polymeric network of hydrogels, they may undergo continuous bonding/debonding under mechanical or thermal stress, which is known as self-healing ability. Self-healing hydrogels may be injectable as the bonds can readily break under shear-induced extrusion through a needle and reform at the injection site.

Although carboxylated chitosan of animal origin has been discovered and investigated in detail in the literature over the past few decades, a recent invention describes a carboxylated chitosan of fungal origin that is soluble at acidic, neutral and alkaline pH values, and proposes the use of this polymer for injection in osteoarthritis synovial joints among other biomedical applications (U.S. 2021/0171667). Nevertheless, the proposed polymer is not cross-linked, which potentially decreases its residence time in the joint. A solution for increasing the residence time of linear polymer chains in biological media is to crosslink them. However, chemical crosslinking of linear polymers typically results in branched polymers with inferior lubricating properties compared to their linear counterparts. Therefore, to obtain a cross-linked polymer with dynamic crosslinking that do not negatively affect lubricating properties of polymers is a significant technological challenge.

It is thus desired to be provided with new lubricating formulation.

### SUMMARY

It is provided a lubricating hydrogel composition comprising an amine-bearing biopolymer, and an oxidized aldehyde-bearing polysaccharide, wherein the aldehyde groups of the aldehyde-bearing polysaccharide are reversibly cross-linked to the amine groups of the amine-bearing biopolymer.

In an embodiment, the amine-bearing biopolymer is chitosan, carboxylated chitosan, glycosylated-chitosan, collagen or gelatin.

In another embodiment, the oxidized aldehyde-bearing polysaccharide is glycogen, phytoglycogen, cellulose nanocrystal, cellulose nanofibers, dextran, or starch.

In a further embodiment, the composition comprises carboxylated chitosan and sweet-corn derived phytoglycogen.

In a supplemental embodiment, the amine-bearing biopolymer is carboxylated chitosan with a molecular weight values in the range 20-500 kg mol⁻¹.

In an embodiment, the aldehyde-bearing polysaccharide is oxidized phytoglycogen from sweet corn with an average hydrodynamic diameter of 30-100 nm, when dispersed in pure deionized water.

In an embodiment, the aldehyde-bearing polysaccharide is oxidized with a degree of oxidation from 0.1 to 1000%.

In a further embodiment, the carboxylated chitosan has a degree of substitution ranging from 10 to 90%.

In another embodiment, wherein the carboxylated chitosan has a degree of deacetylation ranging from 30 to 90%.

In another embodiment, the composition has a pH of 6.5-8.5.

In an embodiment, the composition described herein further comprises an electrolyte and a buffer, including phosphate buffered saline (PBS) and 4-(2-hydroxyethyl)-1-piperazineethanesulfonic acid (HEPES), preferably cells, stem cells, peptides, proteins, growth factors, platelet-rich plasma, bone-derived materials, calcium phosphate, calcium carbonate, or pharmaceutical small molecules peptides, alternatively mannitol, sorbitol, glutathione, or acetaminophen.

In an embodiment, the lubricating hydrogel composition described herein is formulated for an injection in synovial joints, as an injectable dermal filler or as a cosmetic ingredient.

It is further provided a pharmaceutical composition comprising the lubricating hydrogel composition as described herein and a drug or a medical device.

In an additional embodiment, the lubricating hydrogel composition as described herein is for treating osteoarthritis and cartilage repair.

### BRIEF DESCRIPTION OF THE DRAWINGS

Reference will now be made to the accompanying drawings.

FIG. 1a shows chemical formulation of carboxylated chitosan.

FIG. 1b shows chemical formulation of (i) glycogen and (ii) a chemical formulation of a glucose monomer before and after oxidation using periodate ions.

FIG. 1c schematically shows crosslinking of carboxylated chitosan by oxidized glycogen as described herein.

FIG. 2 shows ¹H NMR spectrum of the carboxylated chitosan that was used for hydrogel preparation in accordance to an embodiment.

FIG. 3 shows ¹H NMR spectra for glycogen and oxidized glycogen. The spectrum for oxidized glycogen changes if the oxidation is done in a controlled or uncontrolled manner.

FIG. 4 shows the size distribution of glycogen and oxidized glycogen samples with different degrees of oxidation 1, 3, 5 and 10%. The sample with 5% oxidation, A5, was synthesized in uncontrolled environment as well, resulting on wider size distribution because of nanoparticle degradation.

FIG. 5 shows ¹H NMR spectra for glycogen and oxidized glycogen with different degrees of oxidation 1, 3, 5 and 10%. The inset shows the peak related to the aldehyde group on the polymer chain.

FIG. 6 shows an injectable hydrogel of carboxylated chitosan and aldehyde-modified phytoglycogen in accordance to an embodiment. The carboxylated chitosan with molecular mass of 150 kg mol¹ is 38% carboxylated, while phytoglycogen is 1% aldehyde-modified.

FIG. 7 (left) shows the interaction force profile between two mica surfaces coated with three lubricating formulations Synvisc-One®, Monovisc® and the hydrogel encompassed herein (“present invention”). The radius of curvature, R, for these experiments was 2 cm. The cartoons in the right illustrates the behavior of adsorbed polymer films from each formulation under compression.

FIG. 8 (left) shows the friction force versus normal load profile for two mica surfaces coated with three lubricating formulations Synvisc-One®, Monovisc® and the hydrogel described herein and in accordance to an embodiment (“present invention”). The cartoons in the right illustrates how the adsorbed polymer films protect surfaces against frictional wear. Images show fringes of equal chromatic order (FECO), which directly translate to the shape and condition of the contact area between the compressed surfaces.

### DETAILED DESCRIPTION

In accordance with the present disclosure, there is provided a lubricating hydrogel composition comprising an amine-bearing biopolymer, and an oxidized aldehyde-bearing polysaccharide, wherein the aldehyde groups of the aldehyde-bearing polysaccharide are reversibly cross-linked to the amine groups of the amine-bearing biopolymer.

It is provided a lubricating injectable hydrogel formulation based on carboxylated chitosan and lightly oxidized glycogen, at which glycogen nanoparticles serve as multifunctional crosslinking agents for the carboxylated chitosan chains. The highly hydrated nature of both polymers, as well as the dynamic and reversible nature of the crosslinking junctions, ensure that the resulting hydrogel is an ideal candidate as an injectable joint lubricant. A controlled chemical synthesis protocol ensures that the polymer chains of glycogen nanoparticles do not undergo chemical degradation during oxidation process. Therefore, the NPs retain their shape, size and microstructure, which is essential for their super-lubricating ability.

It is described herein a novel class of self-healing hydrogels made of lightly oxidized plant- or animal-based glycogen polymer and amine-bearing biopolymers carrying various functional groups. The provided self-healing hydrogel is made of a mixture of an amine-bearing biopolymer and an aldehyde-bearing polysaccharide.

In an embodiment, the amine-bearing biopolymer is one or a mixture of the following polymers or their derivatives either from an animal or plant origin: chitosan, carboxylated chitosan, glycosylated-chitosan, collagen or gelatin.

In another embodiment, the aldehyde-bearing polysaccharide is an oxidized version of one or a mixture of the following polymers: glycogen of animal origin, phytoglycogen (glycogen of plant origin), cellulose nanocrystal, cellulose nanofibers, dextran, or starch.

In another embodiment, the amine-bearing biopolymer is a carboxylated chitosan with different molecular weight values in the range 20-500 kg mol⁻¹ and the aldehyde-bearing polysaccharide is oxidized phytoglycogen from sweet corn with an average hydrodynamic diameter of 60-90 nm, when dispersed in pure deionized water.

To synthesize the aldehyde-bearing nanoparticles, oxidation of glycogen is conducted in a controlled condition to ensure that size, shape and nanostructure of nanoparticles remain unchanged. Therefore, the measurement of hydrodynamic diameter before and after oxidation step reflects the same nanoparticle size. In addition, by limiting the oxidation to less than 5% of glucose monomers in glycogen, it is ensured that only monomers on the surface of the nanoparticle are oxidized.

The reversible nature of the crosslinking junctions between aldehyde groups on the surface of nanoparticles and amine groups on linear polymer chains allows them to break under mechanical stress of injection, and reform at the injection site. Therefore, the hydrogels are injectable and self-healing.

In an embodiment, the carboxylated chitosan with animal origin has a degree of acetylation of less than 5%, molecular mass of 100-300 kg mol⁻¹, and degree of substitution of carboxyl groups of 30-50 mol %. In addition, the sweet-corn derived phytoglycogen has a degree of oxidation of 1% or less. The mixture forms an injectable and soft hydrogel that is suitable for injection to synovial joints as a joint lubricant.

In an embodiment, the injectable hydrogel described herein can be used for treating inflammatory joint conditions, osteoarthritis and cartilage defects.

In an embodiment, the hydrogel described herein may be used as a drug delivery vehicle for embedding small therapeutic molecules, peptides, proteins, nanoparticles, and macromolecules, either conjugated chemically or physically to glycogen or chitosan, and their subsequent release in human body.

In another embodiment, the hydrogel is used as an injectable cell scaffold for tissue engineering, cell culture and cell-based therapies.

In another embodiment, the hydrogel used as an injectable dermal filler for cosmetics and pharmaceutical purposes.

In another embodiment, the hydrogel is used as wound healing patches for cosmetics and pharmaceutical purposes.

Accordingly, it is described an hydrogel composed of a mixture of carboxylated chitosan from animal origin as an amine-bearing biopolymer and lightly oxidized sweet-corn derived phytoglycogen as an aldehyde-bearing polysaccharide. The reversible nature of crosslinking from amine-aldehyde interaction grants a hydrogel with self-healing and injectable properties. The exceptionally hydrophilic nature of both polysaccharides and unique nanostructure of spherical glycogen provides the hydrogel with water retention and lubrication properties that make the hydrogel an exceptional candidate for joint lubrication through intra articular injection.

The term hydrogel refers to water-swollen networks of hydrophilic polymers that are cross-linked by covalent or non-covalent bonds to form a 3-dimensional structure that is capable of retaining water at high weight percentages.

The term carboxylated chitosan refers to polysaccharides of D-glucosamine and N-acetyl-D-glucosamine for which part or all of amine groups are permanently and irreversibly substituted by chemical groups containing carboxyl groups. Degree of substitution refers to the molar ratio of carboxyl groups to all the monomers in the chitosan backbone.

Nanotribology experiments (Example III) revealed the lubricating and wear-protecting capability of the disclosed hydrogel (“present invention”) at the molecular scale, which were superior to currently marketed visco supplements such as Synvisc-One® or Monovisc®.

The friction coefficient of the mica surfaces coated with the hydrogel described here was 10⁻², which is equivalent to the friction coefficient in healthy human joints. Whereas, the friction coefficient of the surfaces coated with hyaluronic-based formulations were of the order of 10⁻¹, which is indicative of a poorly lubricated surface.

A nanoscale adsorption test (Example III and FIG. 7B) indicated that the encompassed hydrogel adsorbs and protects negatively charged surfaces against frictional wear, unlike hyaluronic-based formulations such as Synvisc-One® and Monovisc®.

In an embodiment, the degree of deacetylation, which is the ratio of D-glucosamine monomers to all the monomers in the polymer varies between 90-99%.

In another embodiment, the degree of deacetylation varies between 30-90%, by reacetylation of chitosan.

In an embodiment, the degree of substitution varies between 30-60%, and the resulting polymer is still soluble despite high degrees of deacetylation (>95%).

In an embodiment, the amine-bearing biopolymer is carboxylated chitosan with weight-average molecular mass of 20-100 kg mol⁻¹.

In another embodiment, the amine-bearing biopolymer is carboxylated chitosan with weight-average molecular mass of 100-300 kg mol⁻¹.

In another embodiment, the amine-bearing biopolymer is carboxylated chitosan with weight-average molecular mass of 300-500 kg mol⁻¹.

The molecular mass of the carboxylated chitosan decreases by up to 40% during the steam sterilization process at 121° C. for 30 minutes.

The term oxidized polysaccharide or aldehyde-bearing polysaccharide refers to a polysaccharide that has been reacted with an oxidizing agent such as periodate ions to introduce aldehyde groups to the macromolecule.

In another embodiment, the aldehyde-bearing polysaccharide is oxidized phytoglycogen from sweet corn with hydrodynamic diameter of 60-80 nm, when dispersed in pure deionized water.

In another embodiment, the aldehyde-bearing polysaccharide is oxidized glycogen from animal or plant source with hydrodynamic diameter of 30-100 nm, when dispersed in pure deionized water.

In another embodiment, the aldehyde-bearing polysaccharide is oxidized in a way that the percentage of the glucose monomers that were oxidized is within the range of 0.1-1%.

In another embodiment, the aldehyde-bearing polysaccharide is oxidized in a way that the percentage of the glucose monomers that were oxidized is within the range of 1-5%.

In another embodiment, the aldehyde-bearing polysaccharide is oxidized in a way that the percentage of the glucose monomers that were oxidized is within the range of 5-10%.

In another embodiment, the aldehyde-bearing polysaccharide is oxidized in a way that the percentage of the glucose monomers that were oxidized is within the range of 10-50%.

In another embodiment, the aldehyde-bearing polysaccharide is oxidized in a way that the percentage of the glucose monomers that were oxidized is within the range of 50-100%.

The oxidation of the polysaccharide may be done by several oxidizing agents, including but not limited to periodate, hypochlorite, ozone, peroxides, hydroperoxides, persulphates and percarbonates. In an embodiment, the oxidizing agent is sodium periodate for oxidation of glycogen as described by Bertoldo et al. (2013, Polymer Chemistry, 4:653-661).

The encompassed hydrogel is made of the mixture of said chemically modified polysaccharides, is stable in buffer solutions such as phosphate buffer saline that has similar ionic components and pH compared to biological fluids such as synovial fluid and blood serum.

It is further encompassed that the hydrogel may contain various additives depending on the applications. These include, but not limited to, one component from the group consisting of cells, stem cells, peptides and proteins, growth factors, platelet-rich plasma, bone-derived materials, calcium phosphate, calcium carbonate, pharmaceutical small molecules. The additives may be chemically or physically conjugated to the polymers or freely roam inside the hydrogel without interacting with the polymers.

When compared to commercially available formulations, Synvisc-One® and Monovisc®, which do not adsorb strongly to negatively-charged surfaces as they only carry negatively-charged functional groups, the polymer mixture as described herein, showed a strong adsorption layer and upon compression, this layer dehydrated partially and the thickness of the adsorbed layer decreased slightly, making a formulation performing better than known lubricant. Furthermore, the hydrogel described herein forms a protective hydration layer on negatively charged surfaces, providing the surface with lubrication properties similar to that in the healthy human joints and protect the surface against frictional wear.

### Example I

### Synthesis of Carboxylated Chitosan

The purpose of this example is to describe the synthesis protocol used for preparation of carboxylated chitosan, which is a hydrophilic amine-bearing biopolymer that can be used for preparation of super lubricating injectable hydrogels for joint lubrication.

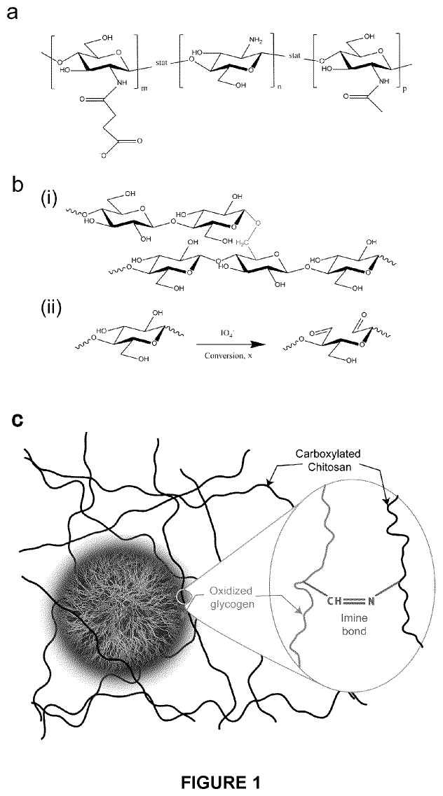

The synthesis protocol is modified compared to that previously described (U.S. Pat. No. 9,901,543). Briefly, the chitosan was deacetylated first to achieve a high degrees of deacetylation (>95%). Deacetylation of chitosan is performed by dispersing chitosan in a 12.5 M sodium hydroxide solution and subsequent steam sterilization at 121° C. for 60 minutes. The chitosan powder is then filtered and rinsed with pure water until a pH of 6-7 is reached. Finally, the chitosan powder is filtered and lyophilized to remove moisture. The chitosan was then dissolved in acidic water and the pH was subsequently adjusted to 5.5. Succinic anhydride (SA) powder was added to the chitosan solution gradually and slowly to a molar ratio of 1:2 for SA:NH₃, while maintaining the pH at 5.5-6 by concurrent addition of a sodium bicarbonate solution. The gradual addition of SA and sodium bicarbonate was done to generate unprotonated amine groups without polymer precipitation. The pH was eventually increased to 8 by addition of sodium bicarbonate. The polymer was then purified from unreacted chemicals and salts using tangential flow filtration, precipitated in 2-proponal, lyophilized and grinded. The chemical formulation of the resulting polymer is shown in FIG. 1a.

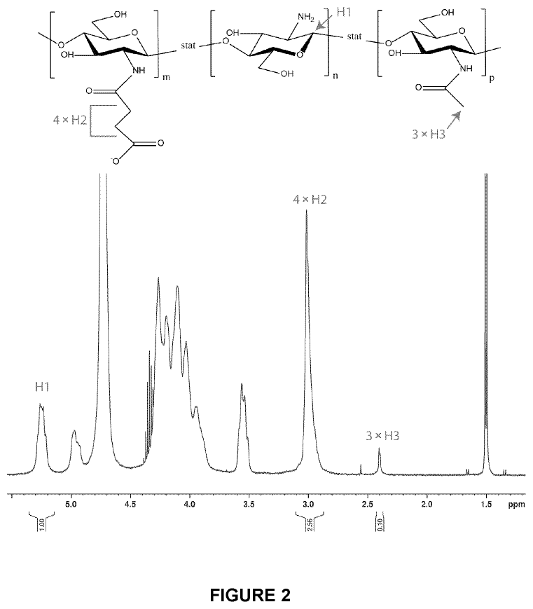

The ¹H NMR spectrum of the polymer was obtained by dissolving the polymer in a mixture of deuterium oxide and deuterium chloride as shown in FIG. 2. The degree of deacetylation (DDA) of 98% and the degree of substitution (DS) of 38% were calculated according to the following equations:

$$DDA = \frac{{H}_{1} + {H}_{2}}{{H}_{1} + {H}_{2} + {H}_{3}}$$

$$DS = \frac{{H}_{2}}{{H}_{1} + {H}_{2} + {H}_{3}}$$

### Example II

### Synthesis of Aldehyde-Bearing Glycogen

Oxidized sweet-corn derived phytoglycogen was synthesized following a similar protocol reported by Bertoldo et al. (supra) for synthesis of oxidized animal-derived glycogen. Briefly, phytoglycogen was dispersed in pure water at a concentration of 5 w % for 2 hours. The resulting dispersion contains monodispersed phytoglycogen nanoparticles with an average hydrodynamic diameter of 75 nm. The dispersion was then covered in aluminum foils and was kept in an ice-bath for subsequent oxidation steps. A solution of sodium periodate was prepared and was added to phytoglycogen dispersion to achieve a molar ratio of 1, 3, 5 or 10% for periodate ions to glucose monomers in glycogen. The mixture was kept agitated in ice bath for 30 minutes. Finally, ethylene glycol was added to quench the reaction with a molar concentration equivalent to 10 times the molar concentration of added periodate ions. The dispersion was dialyzed against pure water for five days with water exchange every day.

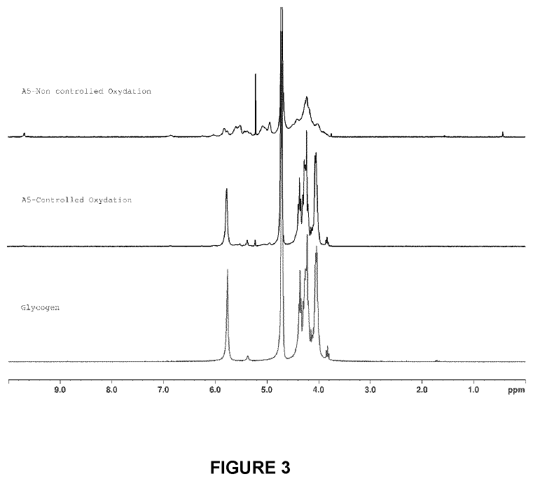

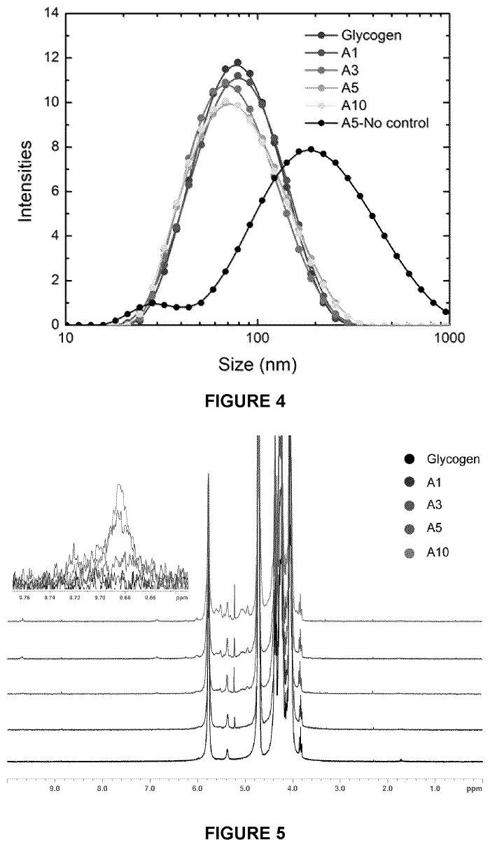

The chemical structure of phytoglycogen, glucose monomers and oxidized glucose monomers are shown in FIG. 1b. The oxidation reaction is sensitive to temperature and light. Conducting the same synthesis protocol that is described here, without maintaining the dark condition and the temperature at 0° C. results in decomposed oxidized nanoparticles. For example, FIG. 3 shows that oxidizing 5% of glucose monomers in phytoglycogen, with and without control of temperature results in a significantly different results, with more side reactions and chemical species being produced without control of temperature and light. This was also evident from dynamic light scattering measurement that are shown in FIG. 4. While the oxidized samples that were prepared at dark condition and 0° C. did not show any significant size differences compared to intact phytoglycogen, the sample that was prepared at room temperature showed a significant variation in size distribution, suggesting nanoparticle decomposition under oxidation. Despite that the size of the oxidized nanoparticles do not change under controlled conditions, FIG. 5 shows that the peak at chemical shift of 9.69 ppm, which is attributed to aldehyde groups on oxidized phytoglycogen, enhances with increase in oxidation agent, suggesting higher quantity of aldehyde without nanoparticle decomposition.

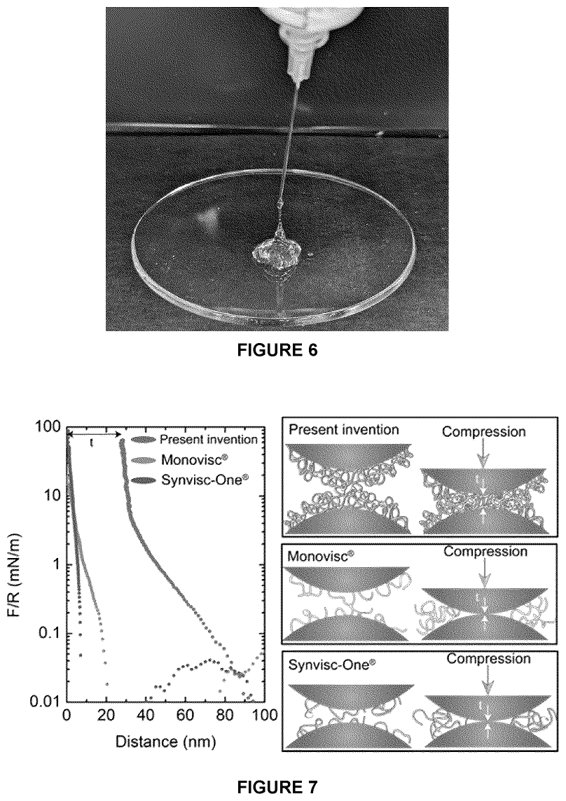

Hydrogels can be prepared by mixing the carboxylated chitosan that was described in Example I with oxidized glycogen even with the lowest amount of aldehyde modification described in this example. For example, FIG. 6 shows an injectable hydrogel made of a mixture of 1 w % carboxylated chitosan and 0.1 w % oxidized phytoglycogen with 1% monomer oxidation in phosphate buffer saline (PBS).

### Example III

### Lubrication of Negatively-Charged Solid Surfaces

Interaction of macromolecules with negatively-charged surfaces is of significant importance for materials that are injected to biological media because most biological surfaces, such as cartilage surface and cell membranes carry a net negative charge. In this example, it is demonstrated that the hydrogel described in the present art is an effective lubricant of negatively charged surfaces, unlike hyaluronic-based formulations. For this example, a nanotribology setup was employed that can detect polymers chain dynamics close to surfaces at the molecular scales. Therefore, it is possible to infer how polymer chains are distributed on solid surfaces, and how this distribution is affected by compressive and shear stress.

The performance of the hydrogel described in the present art was compared to two commercially available visco supplements, namely Synvisc-One® and Monovisc®, the former representing chemically cross-linked hyaluronic acid formulations and the latter representing formulations with linear chains of hyaluronic acid.

The instrument that was used to characterize the adsorption and lubrication properties of these formulations is the surface force apparatus (SFA), which operates based on multiple-beam interferometry and contact mechanics, and is widely used to study intermolecular and surface forces in soft matter. The surfaces that are used in these experiments are made of mica, which are negatively-charged atomically-flat surfaces.

As the first step, adsorption of different formulations was investigated on mica surfaces, which demonstrates if the adsorbed films of these formulations can protect the surfaces under compressive stress. For this purpose, a diluted sample of these three formulations (each containing 20 μg mL⁻¹ polymer in PBS) was deposited on the surfaces and left unperturbed for 1 hour. Following the adsorption step, the surfaces were gently rinsed by PBS to remove non-adsorbed polymer. The surfaces that were modified by the polymer coating were them mounted on the SFA to obtain the normal force profile. Therefore, the surfaces were approached and compressed against each other while measuring the normal force. The data from this experiment is shown in FIG. 7 (left). For the polymer mixture as described herein, an adsorbed layer of hydrated polymer was detected at 100 nm separation distance, suggesting a 50 nm adsorbed layer on each surface. Upon compression, this layer dehydrated partially and the thickness of the adsorbed layer decreased to approximately 15 nm on each surface. On the other hand, for the cross-linked and linear hyaluronic acid formulations, Synvisc-One® and Monovisc® respectively, the hydrated adsorbed layers were always smaller than 10 nm on each surface, which immediately approached zero under compressive load, suggesting complete removal of the polymer from the surface. This observation was consistent with previous reports that hyaluronic acid does not adsorb strongly to negatively-charged surfaces as they only carry negatively-charged functional groups (Faivre et al., 2017, ACS Nano, 11:1762-1769). This behavior was schematically illustrated in FIG. 7 (right).

The normal force profile demonstrated that the polymer mixture of carboxylated chitosan and oxidized glycogen can form a protective film on negatively-charged solid surfaces, unlike hyaluronic acid. This phenomenon is attributed to the presence of positively charged amine groups on carboxylated chitosan that can electrostatically attach to negatively-charged mica surface. In addition, it has been shown in the literature that the abundance of hydroxyl groups on glycogen nanoparticles causes their adhesion to various surfaces by hydrogen bonding.

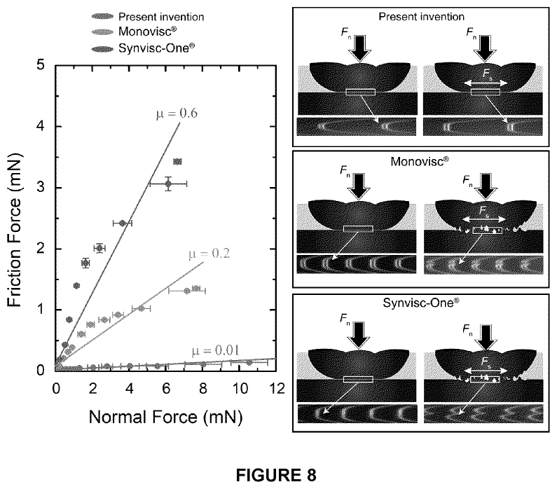

It is further demonstrated how the adsorbed films of different formulations contribute to lubrication properties of the surfaces. In these experiments, the friction force was measured by oscillating the lower surface horizontally at a frequency of 50 mHz, equivalent to a sliding speed of 3 μm s⁻¹, and measuring the resulting friction force from sliding the surfaces past each other. This procedure was done at increasing normal forces to obtain the friction coefficient, which is defined as the ratio of the friction force to normal force. The data for friction force versus the normal force is shown in FIG. 8 (left) for the three formulations. The adsorbed layer of polymer mixture as encompassed herein (“present invention”) resulted in a friction coefficient of 10⁻², which is equivalent to the friction coefficient in healthy human joints. In addition, as shown in FIG. 8 (right), from the images of the fringes of equal chromatic order (FECO), which directly represent the shape of the contact area, the contact area between the compressed surfaces remained unchanged and protected before and after the lubrication experiment. Nevertheless, the friction coefficient of the hyaluronic-based formulations were of the order of 10⁻¹, which is indicative of a poorly lubricated surface. This poor lubrication is also evident from the FECO images, demonstrating that although the contact area between the surfaces were clean and smooth, after applying the sliding motion the contact area became distorted, showing evidence of surface roughness and debris, which is indicative of surface wear. This was also expected as the hyaluronic formulations did not remain adsorbed on the negatively-charged surfaces to be able to protect them against frictional wear.

Overall, this experiment demonstrated that the hydrogel described herein can form a protective hydration layer on negatively charged surfaces, providing the surface with lubrication properties similar to that in the healthy human joints and protect the surface against frictional wear. Unlike this formulation, hyaluronic based formulations do not adsorb to negatively charged surfaces and sliding the surfaces against each other results in frictional wear and damage to the surface.

While the present disclosure has been described in connection with specific embodiments thereof, it will be understood that it is capable of further modifications and this application is intended to cover any variations, uses, or adaptations and including such departures from the present disclosure as come within known or customary practice within the art and as may be applied to the essential features hereinbefore set forth, and as follows in the scope of the appended claims.

1. A lubricating hydrogel composition comprising:
(i) an amine-bearing biopolymer, and
(ii) an oxidized aldehyde-bearing polysaccharide,
wherein the aldehyde groups of the aldehyde-bearing polysaccharide are reversibly cross-linked to the amine groups of the amine-bearing biopolymer.

2. The lubricating hydrogel composition of claim 1, wherein the amine-bearing biopolymer is chitosan, carboxylated chitosan, glycosylated-chitosan, collagen or gelatin.

3. The lubricating hydrogel composition of claim 1, wherein the oxidized aldehyde-bearing polysaccharide is glycogen, phytoglycogen, cellulose nanocrystal, cellulose nanofibers, dextran, or starch.

4. The lubricating hydrogel composition of claim 1, said composition comprising carboxylated chitosan and sweet-corn derived phytoglycogen.

5. The lubricating hydrogel composition of claim 4, wherein the amine-bearing biopolymer is carboxylated chitosan with a molecular weight values in the range 20-500 kg mol⁻¹.

6. The lubricating hydrogel composition of claim 4, wherein the aldehyde-bearing polysaccharide is oxidized phytoglycogen from sweet corn with an average hydrodynamic diameter of 30-100 nm, when dispersed in pure deionized water.

7. The lubricating hydrogel composition of claim 1, wherein the aldehyde-bearing polysaccharide is oxidized with a degree of oxidation from 0.1 to 1000%.

8. The lubricating hydrogel composition of claim 2, wherein the carboxylated chitosan has a degree of substitution ranging from 10 to 90%.

9. The lubricating hydrogel composition of claim 2, wherein the carboxylated chitosan has a degree of deacetylation ranging from 30 to 90%.

10. The lubricating hydrogel composition of claim 1, wherein the composition has a pH of 6.5-8.5.

11. The lubricating hydrogel composition of claim 1, further comprising an electrolyte and a buffer.

12. The lubricating hydrogel composition of claim 1, further comprising cells, stem cells, peptides, proteins, growth factors, platelet-rich plasma, bone-derived materials, calcium phosphate, calcium carbonate, or pharmaceutical small molecules peptides.

13. The lubricating hydrogel composition of claim 1, further comprising mannitol, sorbitol, glutathione, or acetaminophen.

14. The lubricating hydrogel composition of claim 1, formulated for an injection in synovial joints, as an injectable dermal filler or as a cosmetic ingredient.

15. A pharmaceutical composition comprising the lubricating hydrogel composition of claim 1 and a drug or a medical device.

16. A method of treating osteoarthritis and/or cartilage repair in a patient comprising administering to said patient the lubricating hydrogel composition of claim 1.
